(19) United States
(12) Patent Application Publication
(10) Pub. No.: US 2014/0196720 A1
(43) Pub. Date: Jul. 17, 2014

(54) NASAL CUSHION INCLUDING A CONFORTABLE SEPTUM/NARE SEAL

(71) Applicant: Matthew Paul Eury, Latrobe, PA (US)

(72) Inventor: Matthew Paul Eury, Latrobe, PA (US)

(73) Assignee: KONINKLIJKE PHILIPS N.V., EINDHOVEN (NL)

(21) Appl. No.: 14/239,269

(22) PCT Filed: Aug. 9, 2012
(86) PCT No.: PCT/IB2012/054061

§ 371 Date: Feb. 18, 2014

Related U.S. Application Data
(60) Provisional application No. 61/526,753, filed on Aug. 24, 2011.

(51) Int. Cl. A61M 16/06

(52) U.S. Cl. CPC A61M 16/0611
USPC 128/206.24

(57) ABSTRACT

A cushion (16) for use with a mask in delivering a flow of breathing gas to a user includes a first portion (38) adapted to be coupled to a mask shell (22), a second portion (36) adapted to contact a surface of a user, and a wall portion (40) extending between the first portion and the second portion. The second portion includes a septum region (50) and an alare region (52), the septum region being adapted to curl back a first distance (d₁) from the user responsive to the cushion being donned by a user, and the alare region being adapted to curl back a second distance (d₂) from the user responsive to the cushion being donned by a user, the first distance being greater than the second distance.

### CROSS-REFERENCE TO RELATED APPLICATIONS

This patent application claims the priority benefit under 35 U.S.C. §119(e) of U.S. Provisional Application No. 61/526,753 filed on Aug. 24, 2011, the contents of which are herein incorporated by reference.

### BACKGROUND OF THE INVENTION

1. Field of the Invention

The present invention generally relates to a cushion for use on a patient interface device in a pressure support system that supplies a flow of gas to the airway of a patient, and, in particular, to a cushion that includes a septum/nostril/nare sealing portion.

2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation (NIV). It is also known to deliver continuous positive airway pressure (CPAP) or variable airway pressure, which varies with the patient's respiratory cycle, to treat a medical disorder, such as sleep apnea syndrome, in particular, obstructive sleep apnea (OSA), chronic obstructive pulmonary disease (COPD), or congestive heart failure (CHF).

Non-invasive ventilation and pressure support therapies involve the placement of a patient interface device, which is typically a nasal or nasal/oral mask, on the face of a patient to interface the ventilator or pressure support system with the airway of the patient so that a flow of breathing gas can be delivered from the pressure/flow generating device to the airway of the patient. It is known to maintain such masks on the face of a patient by a headgear having upper and lower straps, each having opposite ends threaded through connecting elements provided on the opposite sides and top of a mask.

Typically, patient interface devices include a mask shell having a cushion attached to the shell that contacts the surface of the patient. The mask shell and cushion are held in place by a headgear that wraps around the head of the patient. The mask and headgear form the patient interface assembly. A typical headgear includes flexible, adjustable straps that extend from the mask to attach the mask to the patient.

Because such masks are typically worn for an extended period of time, a variety of concerns must be taken into consideration. For example, in providing CPAP to treat OSA, the patient normally wears the patient interface device all night long while he or she sleeps. One concern in such a situation is that the patient interface device is as comfortable as possible, otherwise the patient may avoid wearing the interface device, defeating the purpose of the prescribed pressure support therapy. It is also important that the interface device provide a tight enough seal against a patient's face without discomfort. A problem arises in that in order for the mask to maintain a seal without any undue gas leaks around the periphery of the mask, the mask may be compressed against the patient's face.

CPAP and other breathing masks traditionally create sealing problems and pressure on a patient's face due to the design used in the flap/cushion that seals the mask to the patient's face. Patients commonly have leak issues at the sealing interface, develop red marks and sores, and complain about the pressure points on the face. In nasal masks, a primary location for such leaks and pressure points occurs on the upper lip of the patient.

### SUMMARY OF THE INVENTION

Accordingly, it is an object of the present invention to provide an improved cushion for use in a patient interface device that overcomes the shortcomings of conventional cushions. As an aspect of the invention, a cushion is provided that comprises a first portion adapted to be coupled to a mask shell, a second portion adapted to contact a surface of a user, and a wall portion extending between the first portion and the second portion. The second portion comprises a septum region and an alare region. The septum region is adapted to curl back a first distance from a user responsive to the cushion being donned by the user and the alare region is adapted to curl back a second distance from the user responsive to the cushion being donned by the user. The first distance being greater than the second distance.

The alare region may be adapted to rest at or about the right or left alar base of a user upon which the cushion is donned.

The alare region may comprise a notched region adapted to be disposed adjacent to, without overlapping any portion of, a nostril of a user when the cushion is donned by a user.

The septum region may comprise a protrusion disposed generally between, and defined in-part by, the notched region. The protrusion being adapted to be disposed adjacent to, and in contact with, the subnasale of a user and extend generally away from the user when the cushion is donned by the user.

The septum region may be bendable from a first, generally linear position prior to the cushion being donned by a user and a second, generally curled position when the cushion has been donned by a user.

The first portion may comprise a mounting member formed separately from, and coupled sealingly to, the cushion.

As another aspect of the invention a patient interface device is provided. The patient interface device comprising a mask shell and a cushion. The mask shell being adapted to receive a flow of breathing gas. The cushion comprising a first portion coupled to the mask shell, a second portion adapted to contact a surface of a user, and a wall portion extending between the first portion and the second portion. The second portion comprising a septum region and an alare region. The septum region being adapted to curl back a first distance from a user responsive to the cushion being donned by the user and the alare region is adapted to curl back a second distance from the user responsive to the cushion being donned by the user. The first distance being greater than the second distance.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention

### BRIEF DESCRIPTION OF THE DRAWINGS

FIG. 1 is a rear perspective view of a patient interface device according to the principles of the present invention shown (schematically) connected to a gas flow/pressure generating system;

FIG. 2 is an isometric view of the cushion of the patient interface device of FIG. 1;

FIG. 3 is a rear, user side, view of the cushion of the patient interface device of FIG. 1;

FIG. 4 is a sectional view of the cushion of the patient interface device of FIG. 1 taken along line 4-4 of FIG. 3;

FIG. 5 is a sectional view of the cushion of the patient interface device of FIG. 1 taken along line 5-5 of FIG. 3;

FIG. 6 is a sectional view of the cushion of the patient interface device of FIG. 1 taken along line 6-6 of FIG. 3;

FIG. 7 is a top view of the cushion of the patient interface device of FIG. 1 shown disposed on the face of a patient;

FIG. 8 is a front view of the cushion of the patient interface device of FIG. 1 shown disposed on the face of a patient;

FIG. 9 is a sectional view of the cushion of FIG. 7 taken along line 9-9 of FIGS. 7 and 8;

FIG. 10 is a sectional view of the cushion of FIG. 7 taken along line 10-10 of FIGS. 7 and 8;

FIG. 11 is a sectional view of the cushion of FIG. 7 taken along line 11-11 of FIGS. 7 and 8;

FIG. 12 is a front view of a prior art cushion shown disposed on the face of a patient; and

FIG. 13 is a plurality of example patient side openings according to the principles of the present invention.

### DETAILED DESCRIPTION OF THE EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

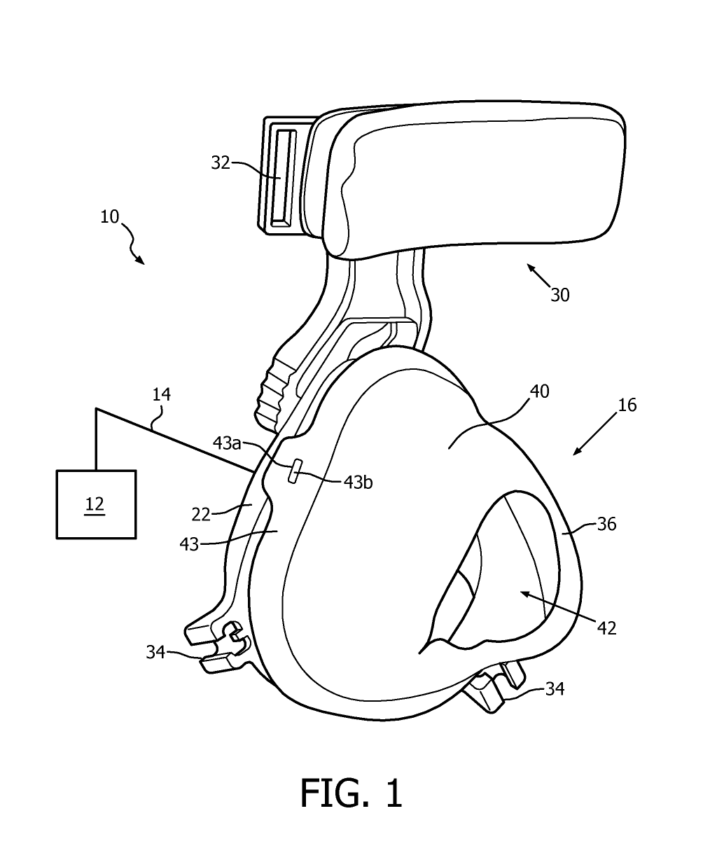

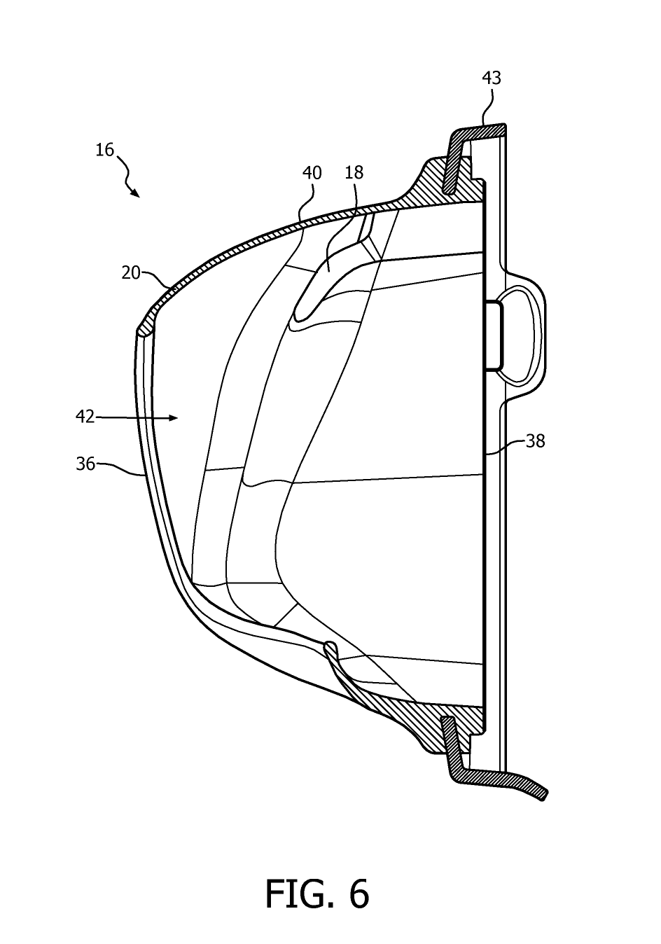

FIGS. 1-11 illustrate an exemplary embodiment of a patient interface device 10 and components thereof according to the principles of the present invention. Patient interface device 10 communicates a flow of breathing gas between the patient's airway and a pressure/flow generating system 12, such as a ventilator, CPAP device, or variable pressure device, e.g., a BiPAP® device manufactured and distributed by Philips Respironics, Inc. of Pittsburgh, Pa., or an auto-titration pressure support system.

A BiPAP® device is a bi-level device in which the pressure provided to the patient varies with the patient's respiratory cycle, so that a higher pressure is delivered during inspiration than during expiration. An auto-titration pressure support system is a system in which the pressure varies with the condition of the patient, such as whether the patient is snoring or experiencing an apnea or hypopnea. For present purposes, pressure/flow generating system 12 is also referred to as a gas flow generating device, because flow results when a pressure gradient is generated. The present invention contemplates that pressure/flow generating system 12 is any conventional system for delivering a flow of gas to an airway of a patient or for elevating a pressure of gas at an airway of the patient, including the pressure support systems summarized above and non-invasive ventilation systems.

Communicating a flow of breathing gas between the patient's airway and pressure/flow generating system 12 includes delivering a flow of breathing gas to the patient from the pressure/flow generating device and exhausting a flow of gas from the patient to ambient atmosphere. The system for delivering a breathing gas to a patient according to the present invention comprises the pressure/flow generating system that produces a flow of gas, and a conduit 14, which is also referred to as a patient circuit, having a first end portion operatively coupled to the gas flow generating device and a second end portion. Conduit 14 carries the flow of gas from pressure/flow generating device 12 during operation of the system to patient interface device 10, which is coupled to the second end portion of the conduit. Conduit 14 corresponds to any conduit suitable for communicating the flow of gas form the pressure/flow generating system to the patient interface device. A typical conduit is a flexible tube. A headgear assembly, which is not shown in the figures, attaches patient interface device 10 to the patient's head.

Patient interface device 10 includes a cushion, generally indicated at 16, and a mask shell 22 having a patient side and opposite thereto, an outer side. Attached to outer side of mask shell 22 is a conduit coupling member (not illustrated) that couples mask shell 22 to conduit 14 so that a flow of gas is communicated to the interior of the patient interface device for subsequent delivery to the patient. Conversely, gas from the patient is communicated from the patient interface device into the conduit, where an exhaust port is located. Mask shell 22 is preferably a generally rigid shell, and, in an exemplary embodiment of the present invention is formed from rigid plastic, such as polycarbonate.

In the illustrated embodiment of FIG. 1, mask shell 22 has a generally triangular shape having a forehead support portion 30 that includes headgear attaching elements in the form of receiving holes or slots 32 disposed on either side of forehead support portion 30 for receiving headgear straps (not illustrated). In the illustrated embodiment, the lower corners of mask shell 22 also include headgear attaching elements in the form of socket attachment elements 34, which cooperate with corresponding ball elements (not illustrated) on headgear straps. The ball and socket configuration, and other headgear attachment configurations suitable for use with the present invention, are disclosed, for example, without limitation, in commonly assigned U.S. Pat. No. 7,066,179, the contents of which are incorporated herein by reference.

It is to be understood that the present invention contemplates using any conventional connection assembly to attach a headgear or headgear strap to mask shell 22 or other suitable shell arrangement. It is to be further understood that the present invention also contemplates eliminating the forehead support entirely, so that the patient interface device is supported on the patient by cushion 16. If the forehead support is eliminated, a headgear attachment may be provided at the upper apex of the mask shell. The present invention also contemplates providing a post or other protrusion at the upper portion of the shell, i.e., the portion overlying the bridge of the nose, to which the headgear can be attached.

The present invention contemplates that the headgear suitable for use with patient interface device 10 is any conventional headgear used in the patient interface field. For example, without limitation, a typical headgear assembly comprises a headpiece that overlies a portion of the patient's crania and with headgear straps extending therefrom to adjustably connect the headgear to the mask.

As perhaps best shown in FIG. 3, cushion 16 has a generally triangular shape and is preferably defined from a unitary piece of soft, cushiony, elastomeric material, such as silicone, appropriately soft thermoplastic elastomers, closed cell foam, thin materials, or any combination of suitable materials. Cushion 16 has a first end portion 36, a second end portion 38 generally opposite the first end portion, and a wall portion 40 extending between first end portion 36 and second end portion 38. A nose receiving cavity 42 is defined in the interior of cushion 16 by wall portion 40.

In the illustrated embodiment, cushion 16 is coupled to mask shell 22 by a mounting member 43 having a shape corresponding generally to that of second end portion 38 of cushion 16. Mounting member 43 includes multiple attachment features 43a, which are adapted to engage corresponding attachment features 43b on mask shell 22. Preferably, mounting member 43 is defined from a relatively rigid material, e.g., without limitation, thermoplastic or other suitable material. The present invention contemplates using any suitable numbers of attachment features and other mechanical or non-mechanical means for attaching the mounting member 43 to mask shell 22. The present invention further contemplates other mechanical or non-mechanical means for attaching cushion 16 to mounting member 43, or for directly attaching cushion 16 to mask shell 22 without the use of mounting member 43, such as by gluing or other suitable means.

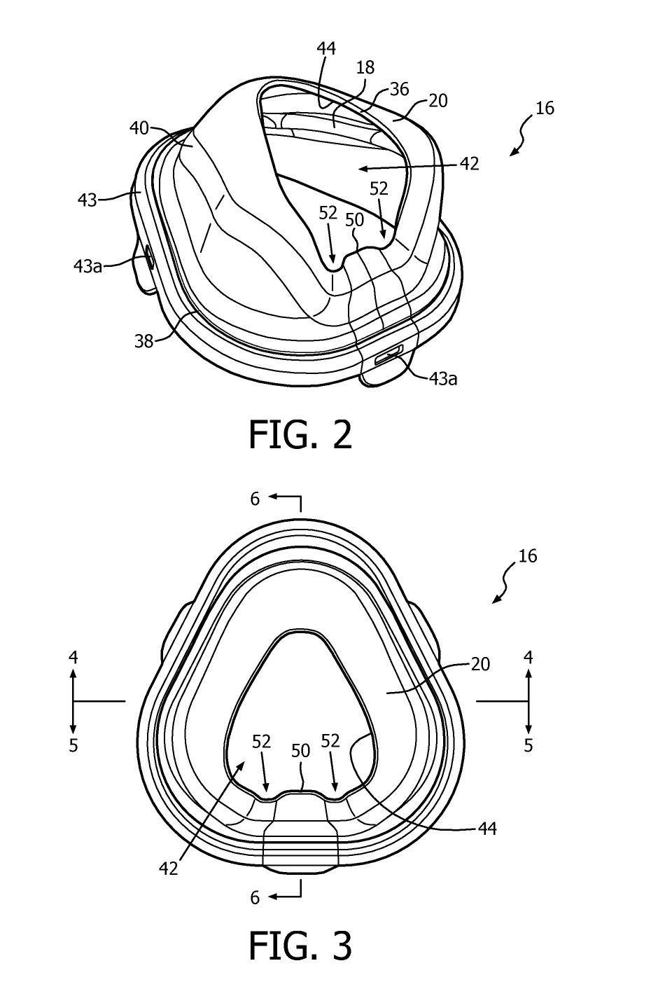

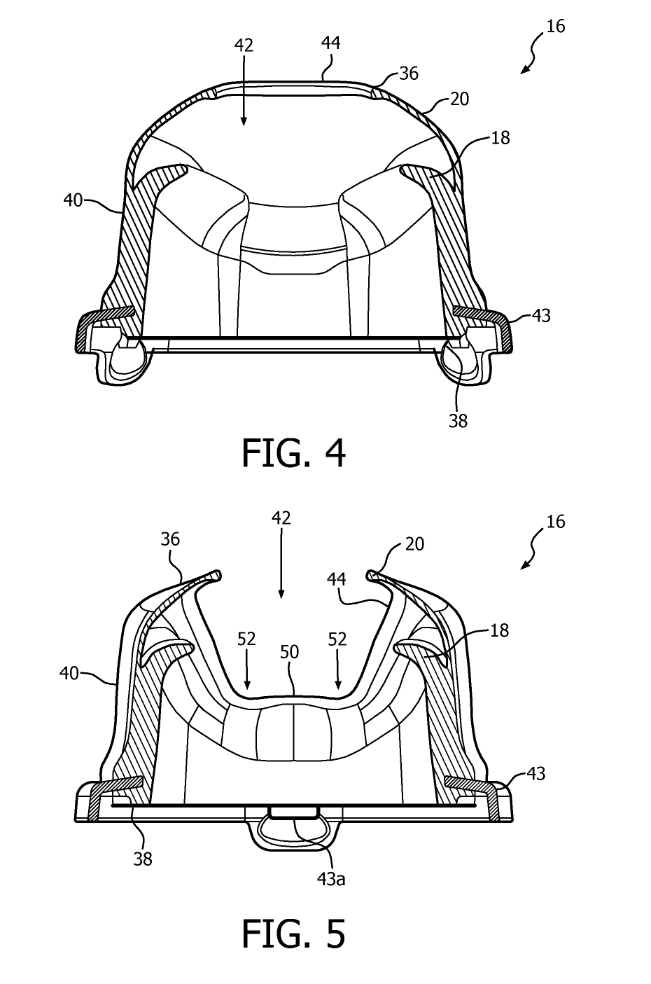

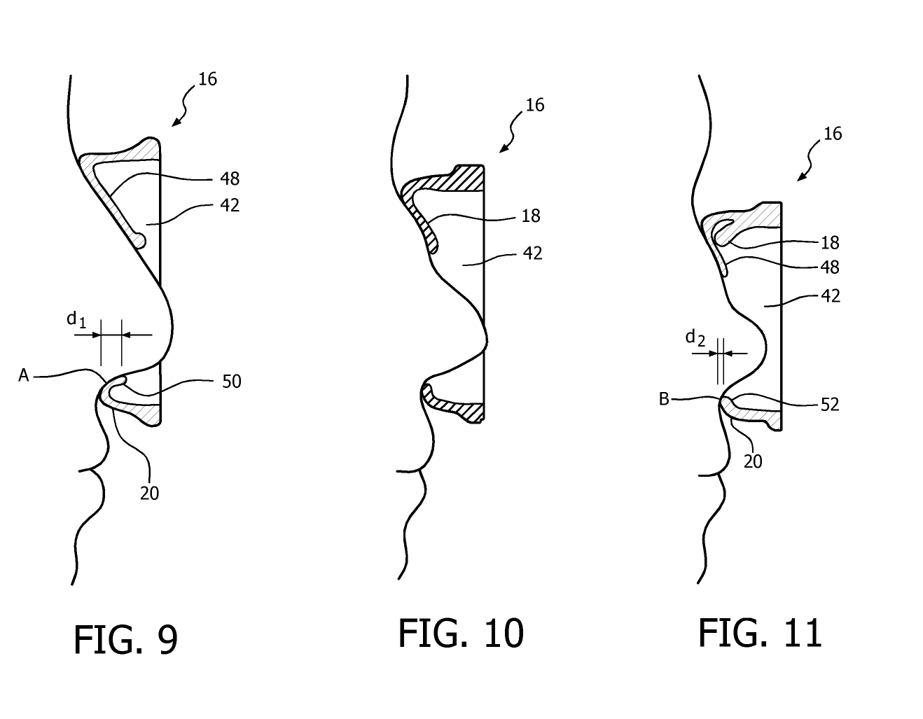

Referring to FIGS. 2-6, first end portion 36, includes a highly flexible sealing flap 20 that is adapted to contact an inner perimeter area of the patient's face and includes a first opening 44 to allow passage of at least a portion of the patient's nose into nose receiving cavity 42, such as shown in the cross-sectional views of FIGS. 9-11, which show an example cushion 16 donned on the head (not numbered) of a user. As perhaps best shown in the cross-sectional view of FIGS. 4 and 5, cushion 16 further includes a pair of somewhat rigid inner support members 18, disposed generally adjacent first end portion 36 and sealing flap 20. It is to be appreciated that such arrangement of first end portion 36 of cushion 16 provides for cushion 16 to be generally supported on either side of the nose of a user via support members 18 and generally sealed with the face of a user via sealing flap 20.

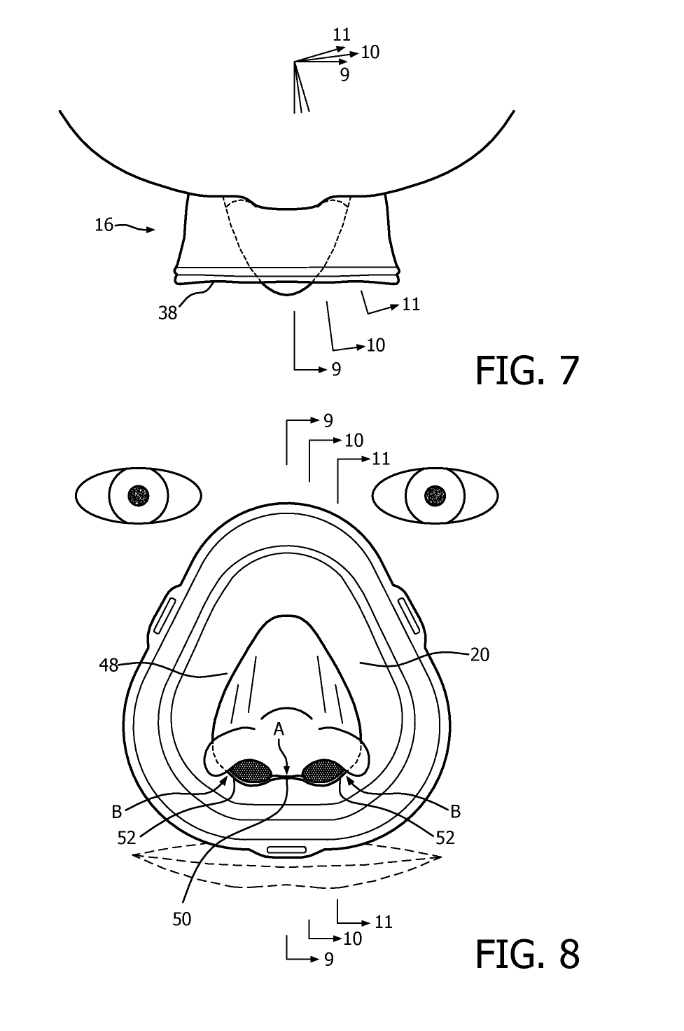

Referring to FIGS. 7-11, when cushion 16 is donned on the head (not numbered) of a user, sealing flap 20 generally curls or bends inward generally away from the face of the user in a manner such that sealing flap 20 contours to match the general facial features of a human. As shown in FIGS. 2, 3, 5 and 8, sealing flap 20 includes an upper, nose bridge engaging region 48, a septum region 50 and an alare regions 52 disposed on either side of septum region 50.

As shown in FIGS. 8-11, when cushion 16, and thus flap 20, is compressed against the face of a user (such as when patient interface device 10 is donned by a user) the sealing technology of cushion 16 wraps around the nasal septum, generally at the subnasale A, and below the right and left alares, generally at or about the right and left alar bases B, while staying below the nostril openings (not numbered) and conforming to them, thus establishing a free and open airpath through the nostrils of the user (see FIG. 8). More particularly, as shown in the cross sectional view of FIG. 9, when donned by a user (not numbered), septum region 50 curls back a distance d₁ from the face of the user in response to contacting the subnasale region A of the user. In comparison, as shown in the cross-sectional view of FIG. 11, alare region 52 curls back a lesser distance d₂, which may be generally negligible and still maintain a satisfactory seal between cushion 16 and the user.

It is to be appreciated that the invention improves seal, reduces sealing contact area, and relieves upper-lip pressure on a user. As shown in FIG. 8, the concave curves of the alare regions 52 on the left and right of septum region 50 create unrestricted openings to the nostrils of the user allowing for unobstructed airflow. The concave curves also help to avoid unwanted bunching or lifting of nose bridge engaging portion 48 of sealing flap 20 thus providing an improved seal that is also more comfortable to a user.

Septum region 50 generally acts to position the curves at the proper position for this unrestricted airflow. Septum region 50 also establishes a position for the maximum distance sealing flap 20/cushion 16 encroaches to the nasal septum functioning as a left-to-right location feature and a stop position in the direction toward the patient. The conforming flap/cushion geometry provides the ability to wear or place the sealing flap closer to the nasal septum (positive “y” direction) because of its contoured shape which requires less sealing surface area on the upper lip, thus resulting in less upper-lip pressure.

It is to be appreciated that the present invention is not limited to the shape of first opening 44 shown in the illustrated embodiment and that other shapes may be employed without varying from the scope of the present invention. For example, FIG. 13 shows a plurality of other example shapes for first opening 44 that may be employed according to the principles of the present invention.

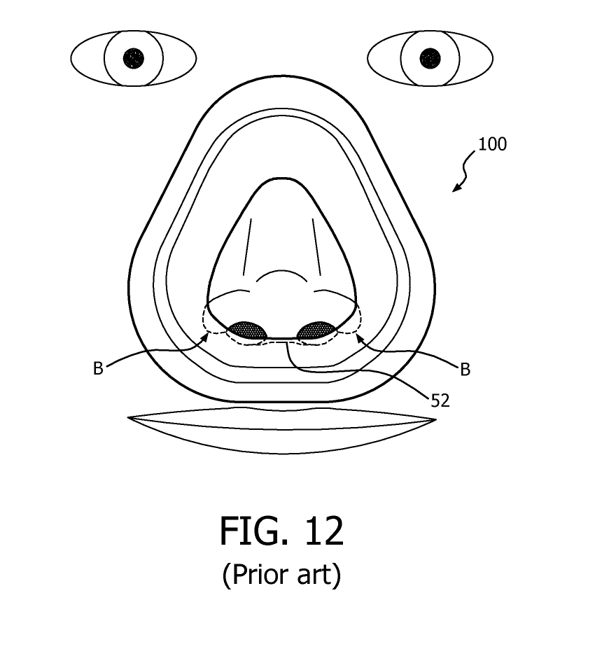

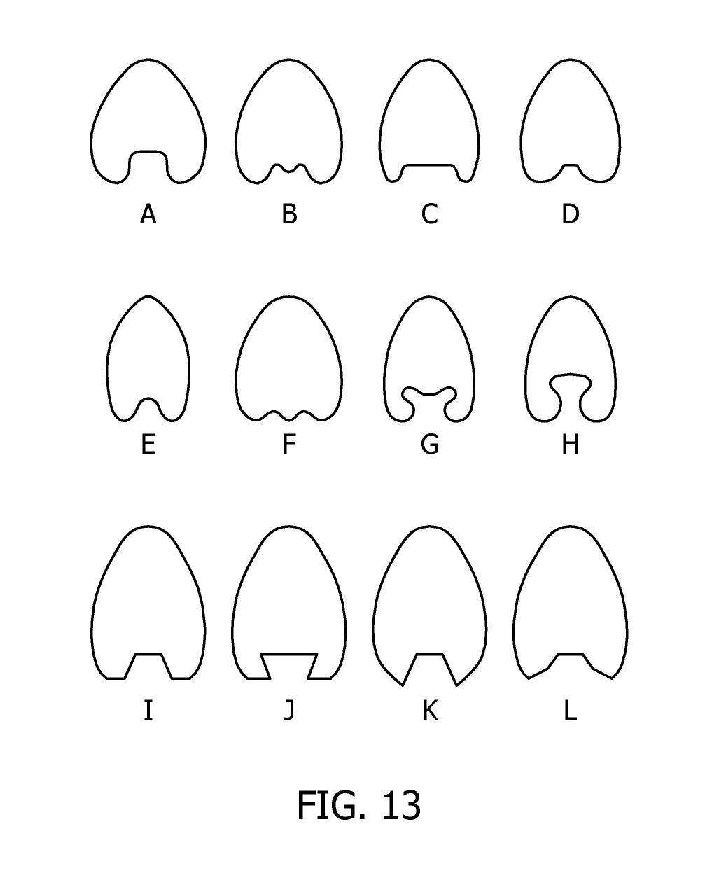

In contrast to the improved cushion 16 disclosed herein, FIG. 12 shows an example prior art cushion 100 disposed on the face of a user. Cushion 100 includes a sealing flap 102 that generally folds upward when cushion 100 is donned on a user. As shown, sealing flap 102 tends to bend in a manner that at least partially blocks one or both nostrils (not numbered) and tends to fold and bunch about the alar bases B. Such folding and bunching generally leads to user discomfort and requires greater pressure applied to cushion 100 (typically via a headgear assembly, not show) to sufficiently seal cushion 100 to the face of the user.

In the illustrated embodiment, cushion 16 is a nasal cushion, meaning that it seals around only the nose of the user. It is to be understood, however, that the present invention contemplates that the 16 may also be used as a portion of patient interface device having a mechanism that engages the mouth of a user.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A cushion comprising:
a first portion adapted to be coupled to a mask shell
a second portion adapted to contact a surface of a user; and
a wall portion extending between the first portion and the second portion, wherein the second portion comprises a septum region and alare regions disposed on either side of the septum region,
wherein each alare region comprises a notched region positioned to be disposed adjacent to, without overlapping any portion of a nostril of the user when the cushion is donned by the user,
wherein the septum region is adapted to curl back a first distance (d₁) from the user responsive to the cushion being donned by the user,
wherein the alare regions are adapted to curl back a second distance (d₂) from the user responsive to the cushion being donned by the user, and
wherein the first distance is greater than the second distance.

2. The cushion of claim 1, wherein the alare regions are adapted to rest at or about the right and left alar base (B) of a user upon which the cushion is donned.

3. (canceled)

4. The cushion of claim 1, wherein the septum region comprises a protrusion disposed generally between, and defined in-part by, the notched regions, the protrusion being adapted to be disposed adjacent to, and in contact with, the subnasale (A) of the user and extend generally away from the user when the cushion is donned by the user.

5. The cushion of claim 1, wherein the septum region is bendable from:
a first, generally linear position prior to the cushion being donned by the user; and
a second, generally curled position when the cushion has been donned by the user.

6. The cushion of claim 1, wherein the first portion comprises a mounting member formed separately from, and coupled sealingly to, the cushion.

7. A patient interface device comprising:
a mask shell adapted to receive a flow of breathing gas; and
a cushion comprising:
a first portion coupled to the mask shell;
a second portion adapted to contact a surface of a user; and
a wall portion extending between the first portion and the second portion, wherein the second portion comprises a septum region and alare regions disposed on either side of the septum region,
wherein each alare region comprises a notched region positioned to be disposed adjacent to, without overlapping any portion of, a nostril of the user when the cushion is donned by the user,
wherein the septum region is adapted to curl back a first distance (d₁) from the user responsive to the cushion being donned by the user,
wherein the alare regions are adapted to curl back a second distance (d₂) from the user responsive to the cushion being donned by the user, and
wherein the first distance is greater than the second distance.

8. The patient interface device of claim 7, wherein the alare regions are adapted to rest at or about the right or left alar base (B) of the user upon which the cushion is donned.

9. (canceled)

10. The patient interface device of claim 7, wherein the septum region comprises a protrusion disposed generally between, and defined in-part by, the notched regions, the protrusion being adapted to be disposed adjacent to, and in contact with, the subnasale (A) of the user and extend generally away from the user when the cushion is donned by the user.

11. The patient interface device of claim 7, wherein the septum region is bendable from:
a first, generally linear position prior to the cushion being donned by the user; and
a second, generally curled position when the cushion has been donned by the user.
